(19) United States
(12) Patent Application Publication
(10) Pub. No.: US 2025/0155329 A1
(43) Pub. Date: May 15, 2025

(54) SAMPLE COLLECTION AND AUTOMATED PROCESSING DEVICE

(71) Applicant: Automated Laboratory Process Solutions, LLC, Las Vegas, NV (US)

(72) Inventors: Gregory D. Ourednik, Chicago, IL (US); Brian Robert Sorel, Statesville, NC (US); Leslie E. Edinboro, Glen Allen, VA (US); Matthew Lee Muhlinghause, Winston-Salem, NC (US)

(21) Appl. No.: 18/507,312

(22) Filed: Nov. 13, 2023

(51) Int. Cl. G01N 1/10

(52) U.S. Cl. CPC G01N 1/10; G01N 2001/1056

(57) ABSTRACT

A device for collection of fluid samples is provided. The device can include a swab for collection of fluids such as oral fluids. The device can further include an expeller configured to expel fluid from the swab to allow processing of the fluids for any desired testing. The swab and expeller can allow more convenient automated processing of samples and can allow efficient collection and use of samples such as oral fluids. It is contemplated that the device may be used for many types of fluids including other bodily fluids besides oral fluids/saliva.

The present disclosure relates generally to devices and methods for collecting fluid samples, such as oral fluids. Also provided are devices to assist in automated laboratory processing of collected samples.

Laboratory processing of various fluid samples is important for a number of diagnostic or investigatory uses. For example, biologic samples, such as oral fluids or other bodily fluids (e.g., nasal discharge, urine, blood or blood components) are useful for evaluating medical conditions, testing for drug use, or other evaluations. However, handling of collected fluid samples can be time consuming, expensive, or potentially hazardous for medical personnel or laboratory workers. Furthermore, fluid samples must be handled properly to prevent contamination of samples and to ensure collection and use of adequate samples volumes.

Accordingly, there is a need for improved systems for collecting and allowing processing of fluid samples, including oral fluids. The systems should provide convenient and rapid means for collecting and transporting samples. Furthermore, the systems should allow for collection and use of sufficient sample volumes and facilitate automated sample handling and processing to address one or more of the aforementioned challenges and requirements for sample collection and processing. The systems can provide advantages such as higher throughput, full automation, and/or less manual handling.

### SUMMARY

The present disclosure provides a device for collection of fluid samples. The device can include a fluid collection device, including an elongated supporting structure, and a fluid collection region disposed at or near a distal end of the supporting structure and including an absorbent material configured to collect fluids that are placed in contact with the absorbent material. The device for collection of fluid samples can further include a holding and expelling component. The holding and expelling component can include a base structure having an outer surface configured to contact a sidewall of a sample collection container, thereby securing the holding and expelling component in the sample collection container. The holding and expelling component can further include a first extension tab extending from a first side of a top portion of the base structure to a first end portion of the first extension tab; and a second extension tab extending from a second side of a top portion of the base structure to a second end portion of the second extension tab. The first end portion and second end portion are arranged to form a gap therebetween, the gap sized to receive a proximal end of the elongated supporting structure to hold the elongated supporting structure. The device for collection of fluid samples can further include a sample collection container.

Also provided is another device for collection of fluid samples. The device can include a fluid collection device, including an elongated supporting structure, and a fluid collection region disposed at or near a distal end of the elongated supporting structure and including an absorbent material configured to collect fluids that are placed in contact with the absorbent material. The device for collection of fluid samples can further include a holding and expelling component. The holding and expelling component can include a base structure having an outer surface configured to contact a sidewall of a sample collection container, thereby securing the holding and expelling component in the sample collection container. The holding and expelling component can further include extension regions extending from the base structure and forming a holding and expelling portion, the holding and expelling portion configured to support a distal end of the elongated supporting structure, and upon pulling the fluid collection device through the holding and expelling portion, applying compressive force to the fluid collection region to cause expulsion of a desired amount of fluid from the absorbent material. The device for collection of fluid samples can further include a sample collection container.

Also provided is a fluid collection device, including an elongated supporting structure, and a fluid collection region disposed at or near a distal end of the supporting structure and including an absorbent material configured to collect fluids that are placed in contact with the absorbent material, wherein the fluid collection device includes a protrusion forming an angled section above the absorbent material.

Also provided is a device for holding and expelling of fluid samples from a collection device. The device can include a holding and expelling component, including a base structure having an outer surface configured to contact a sidewall of a sample collection container, thereby securing the holding and expelling component in the sample collection container. The device can also include a first extension tab extending from a first side of a top portion of the base structure to a first end portion of the first extension tab; and a second extension tab extending from a second side of a top portion of the base structure to a second end portion of the second extension tab. The first end portion and second end portion are arranged to form a gap therebetween, the gap sized to receive a proximal end of the elongated supporting structure to hold the elongated supporting structure.

Also provided are methods of using the aforementioned devices.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 is a side view of a device for collection of fluids, according to certain embodiments.

FIG. 2A is a perspective view of a holding and expelling component of a collection device, according to certain embodiments.

FIG. 2B is a top view of the holding and expelling component of FIG. 2A.

FIG. 2C is a side view of the holding and expelling component of FIGS. 2A and 2B.

FIG. 3A is a perspective view of a portion of a fluid collection device, according to certain embodiments.

FIG. 3B is a side view of the fluid collection device of FIG. 3A.

FIG. 4A is a side view of a device for collecting fluid in a fully assembled state, according to certain embodiments.

FIG. 4B is a side view of the device of FIG. 4A with certain components separated to illustrate use of the device.

FIG. 4C is another side view of the device of FIGS. 4A and 4B with certain components separated to further explain use of the device.

FIG. 4D is another side view of the device of FIGS. 4A-4C illustrating removal of the fluid collection device to illustrate expulsion of collected fluid into a test tube.

FIG. 5 is a side view of a portion of the fluid collection device of FIG. 1 illustrating a proximal portion of the fluid collection device while being held by the holding and expelling component.

FIG. 6 is a side view of a portion of the fluid collection device of FIG. 1 illustrating the fluid collection device while being pulled through the holding and expelling component to expel fluids into the test tube.

FIG. 7 is a perspective view of a fluid collection device, according to various embodiments.

### DETAILED DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purposes.

As stated above, the present disclosure provides devices, components, systems, and methods to facilitate collection, handling, transport, and/or processing of fluid samples. The devices provide convenient and rapid means for collecting and transporting samples. Furthermore, the devices allow for collection and use of sufficient sample volumes and facilitate automated sample handling and processing to address one or more of the aforementioned challenges and requirements for sample collection and processing. The devices can provide advantages such as higher throughput, full automation, and/or less manual handling.

The devices and systems described herein include a fluid collection device and a holding and expelling component. The fluid collection device will generally include an elongate supporting structure with a fluid collecting absorbent component at one end. The fluid collection device can include additional structural features to facilitate reliable operation. The holding and expelling component can be secured in a test tube, or other container or apparatus, to hold the fluid collection device. The holding and expelling component will further include structural features to expel fluid from the collection device into a test tube, other container, or testing apparatus.

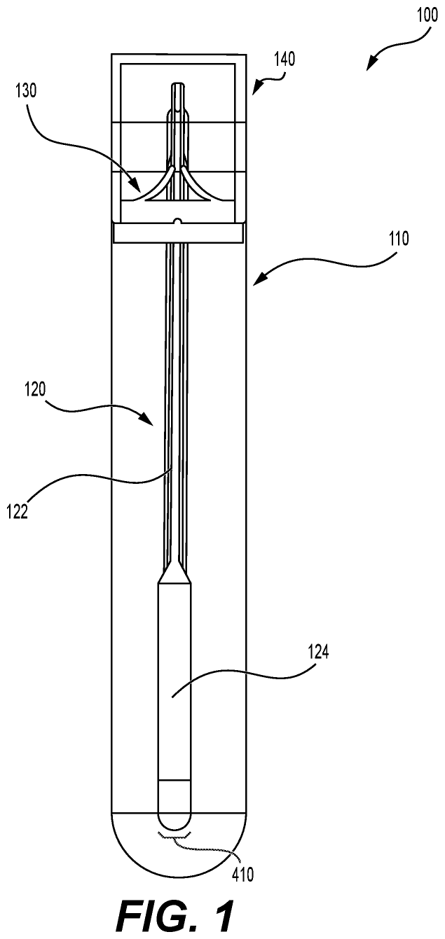

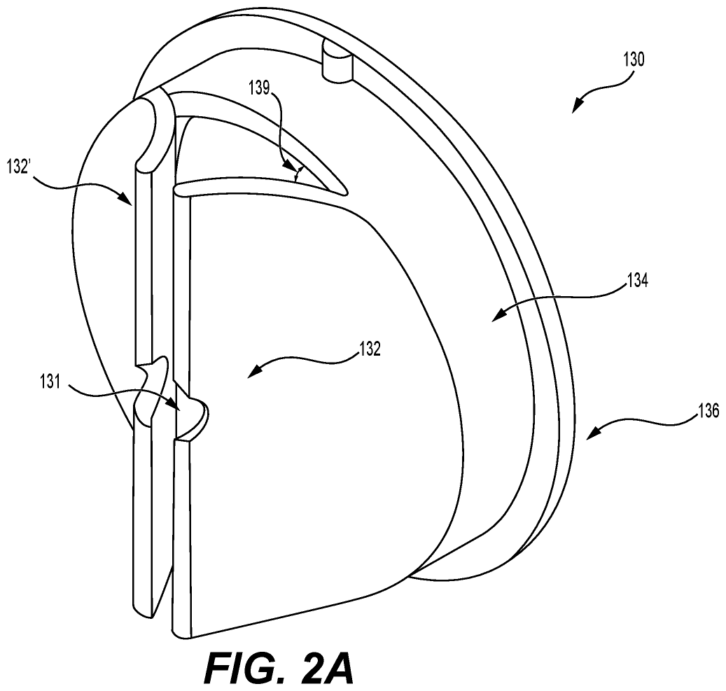

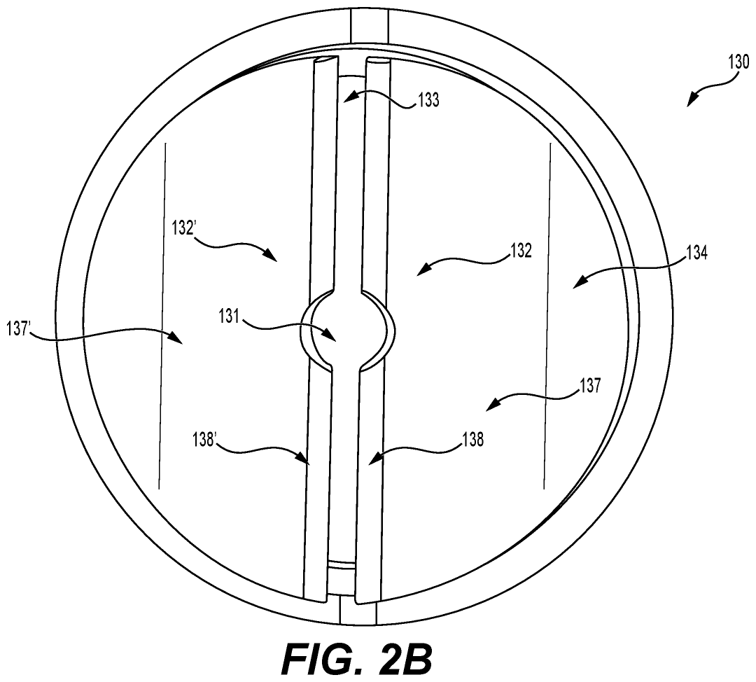

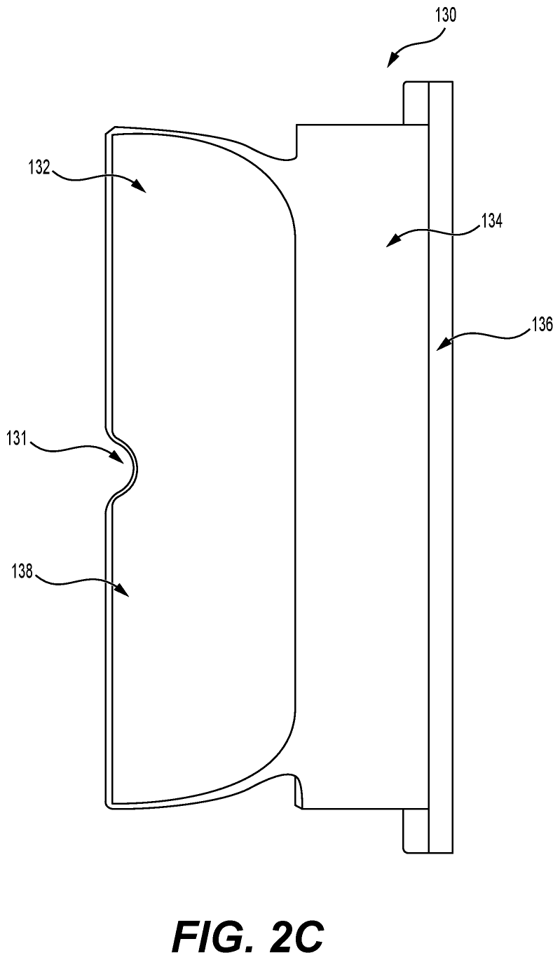

FIG. 1 is a side view of a device 100 for collection of fluids, according to certain embodiments. The device 100 includes a fluid collection device 120 including: an elongated supporting structure 122, and a fluid collection region 124 disposed at or near a distal end of the supporting structure 122 and including an absorbent material configured to collect fluids that are placed in contact with the absorbent material. The device 100 also includes a holding and collecting component 130. As shown in FIGS. 2A-2C, the holding and collecting component 130 includes a base structure 134 having an outer rim or surface 136 configured to contact a sidewall of a sample collection container 110, thereby securing the holding and expelling component in the sample collection container. Generally, the device 100 can include a closure or cap 140 for the container 110. More details on each components are provided further below.

FIG. 2A is a perspective view of a holding and expelling component 130 of a collection device 100, according to certain embodiments. FIG. 2B is a top view of the holding and expelling component 130 of FIG. 2A. FIG. 2C is a side view of the holding and expelling component 130 of FIGS. 2A and 2B. Generally, the component 130 will be sized and shaped to fit into a collection container such as a test tube. Accordingly, the component 130 may be substantially circular when viewed from the top, but other shapes may be used such as ovoid or square shapes. The shape will be defined by a base structure 134, which can include an outer rim or surface 136 sized to fit a test tube or other container.

The holding and expelling component 130 can include structures, e.g., and extension region(s), to secure and hold a portion of the fluid collection device 120 and to expel fluids. In one embodiment, the component 130 includes a first extension tab 132 extending from a first side 137 of top portion of the base structure to a first end portion 138 of the first extension tab 132; and a second extension tab 132′ extending from a second side 137′ of the base structure to a second end portion 138′ of the second extension tab. A gap 133 is formed between the tabs, and the gap 133 can be sized to allow passage of the fluid collection device 120 while applying compressive force to the fluid collection region 124 to expel fluids as the fluid collection device is pulled upward through the holding and expelling component 130.

The holding and expelling component 130 is designed to provide one or more features that allow holding of the collection device 120, allow expulsion or removal of a certain amount or minimum amount of collected fluid, and/or provide for reliable operation of the device (e.g., to ensure consistent expulsion of fluid and error free automated processing. Accordingly, various features of the holding and expelling component 130 are described herein.

The holding and expelling component 130 can be formed of a number of suitable materials. Generally the holding and expelling component 130 will be manufactured as a single material, e.g., as a molded plastic or other similar material. The material may be selected to provide sufficient strength but also a desired degree of flexibility to allow the device to be slightly compressed to snugly fit in a container. Further, the material may provide a certain degree of flexibility to allow application of a desired compressive force to the fluid collection region. Suitable materials can include polyethylene or high density polyethylene, which can be produced with varying degrees of elasticity.

As shown in FIGS. 2A-2B, the gap 133 is substantially linear across the component 130, with the end portions 138, 138′ being generally parallel. Such a configuration provides for a number of advantages and can allow smooth movement with compression of the collection device 120 as it is pulled through the component 130. The gap 133 can have a size (width) dependent on the size of the fluid collection region 124. For example, the gap can have a width of between about 0.25 mm to 5 mm, or about 0.5 mm to 2 mm, or 1 mm to 3 mm, or 1 mm to 2 mm, or 1 to 5 mm, or 1 mm to 4 mm or 0.5 to 2.5 mm. The size of the gap can be varied based on a width of the absorbent material of the fluid collection region 124 and/or elongated supporting structure 120. For example, the gap may be slightly smaller than the width of the absorbent material, such as 90%, 80%, 70%, 60%, 50%, 40%, or 30% of the width of the absorbent material. Further the gap may be configured to be manufactured with a range of widths compared to the thickness of the absorbent material such as 80-90%, 70-90%, 60-80%, 70-80%, 50-90%, 10-90%, 10-30%, 10-40% of the width of the absorbent material.

As an example, a suitable absorbent material may have a thickness (as labeled 410 in FIG. 1) of about 5-15 mm, or more preferably 8-12 mm, or about 9-10 mm. A width of the gap 133 can be 1-5 mm, making the ratio of the absorbent material thickness to width of the gap between about 1:1 to 1:15, or more preferably 1:12 to 8:15, or 1:10 to 3:5. It will be understood that when referring to the absorbent material, the terms “width” or “thickness” mean thickness 410, as shown in FIG. 1

The size of the gap 133 compared to the absorbent material width can be selected to all a certain force applied to the absorbent material or to produce a compressive force to allow expulsion of a certain amount of fluid or a percentage of fluid from the absorbent material. For example, the size of the gap 133, thickness of the absorbent material, and/or physical properties of the tabs 132, 132′ can be selected to produce a compressive force of 8-20 lbs or 9-15 lbs. In some embodiments, the size of the gap 133, thickness of the absorbent material, and/or physical properties of the tabs 132, 132′ can be selected to expel a certain percentage of fluid collected by the fluid collection region 124. For example, the percentage can be at least 30%, at least 40%, at least 50%, or at least 60%. Further, the absorbent material can be configured to absorb a minimum volume of fluid (e.g., 0.5 ml, 1 ml, 1.5 ml, 2 ml, 2.5 ml, or 3 ml) and the percentage can therefore relate to a volume of fluid expelled.

Further, the size of the gap 133, thickness of the absorbent material, and/or physical properties of the tabs 132, 132′ can be selected to control a pull force needed to pull the fluid collection device 100 fluid collection region 124 through the gap. That force may be 2-8 lbs, 3-7 lbs, or 3-5 lbs as examples. Such forces can be consistent with normal human use or applicable to automated machinery.

As shown in FIG. 2A, the tabs 132, 132′ extend upward at an angle 139. The angle can be, for example, 20-60 degrees, but the angle may change based on the material used to produce the various components as well as the desired compressive force to be applied. Further, as shown, the tabs are curved, and the amount of curvature can be varied to change the contact angle between the tabs and the collection device 120.

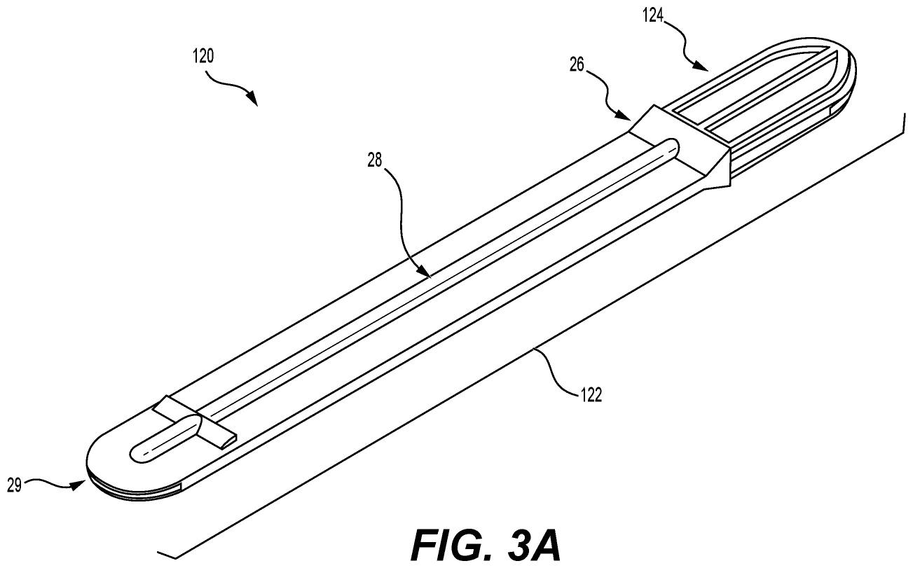

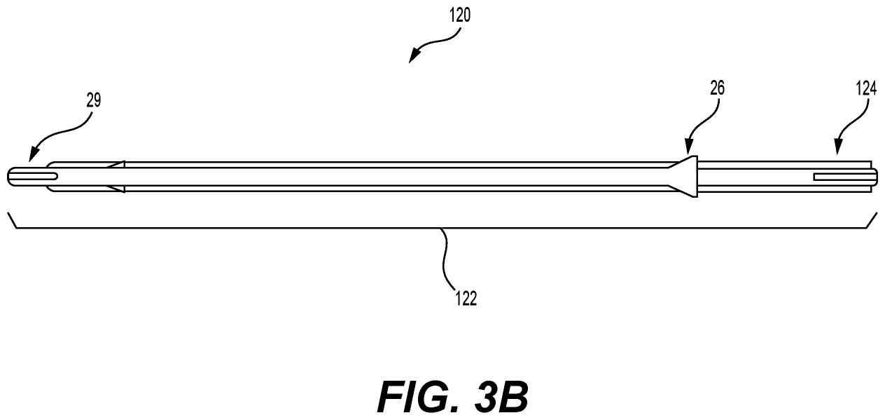

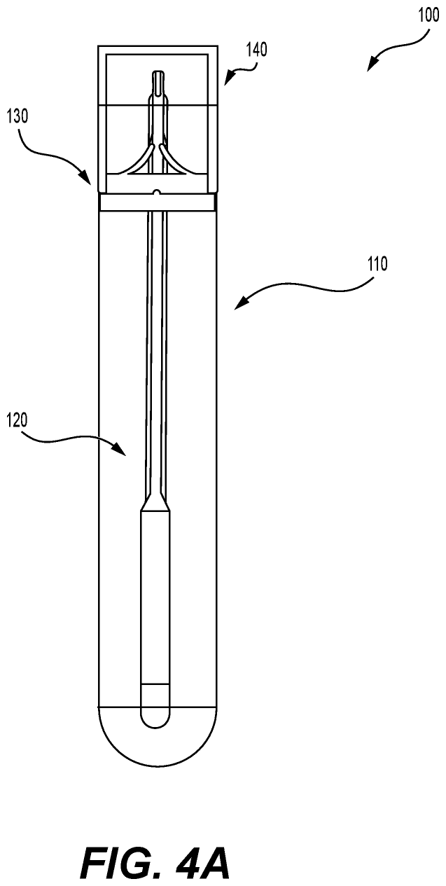

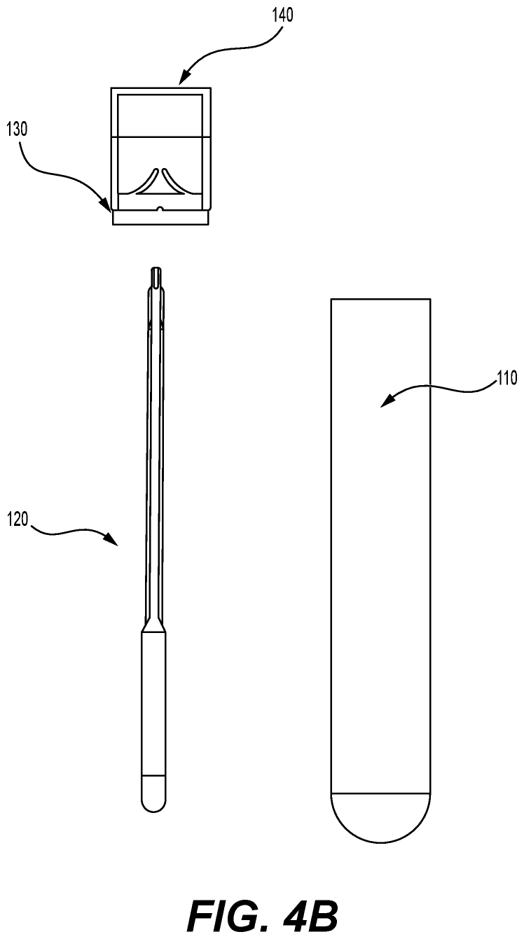

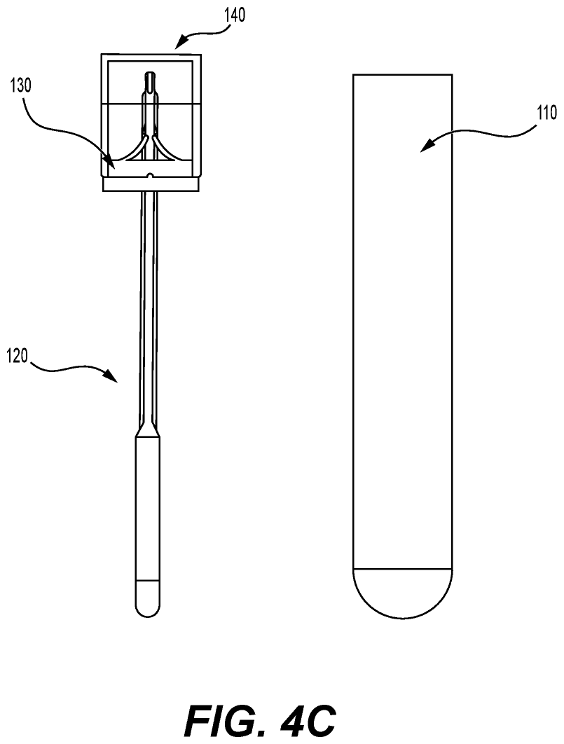

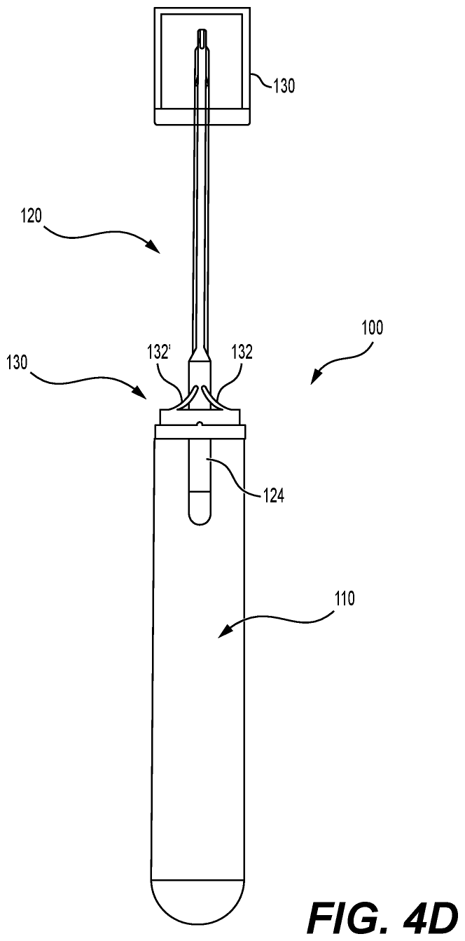

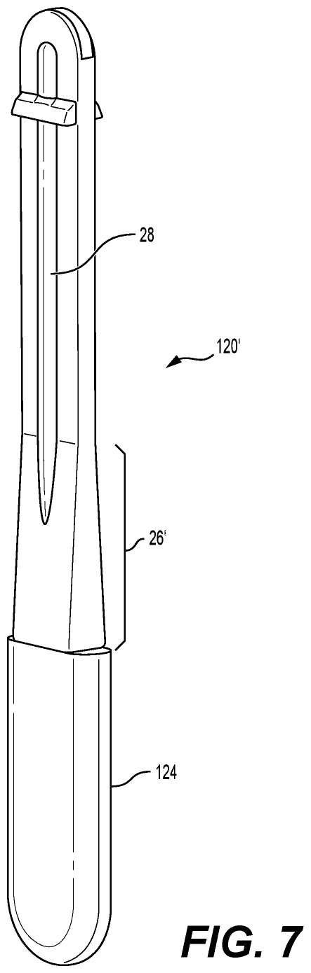

The fluid collection device 120 also includes a number of features to facilitate collection, expulsion, and/or processing of sample. FIG. 3A is a perspective view of a fluid collection device 120, according to certain embodiments. FIG. 3B is a side view of the fluid collection device 120 of FIG. 3A. As shown, the device 120 includes an elongated supporting structure 122, and a fluid collection region 124. The fluid collection region includes an absorbent material (not shown in FIGS. 3A-3B, but shown in FIGS. 4A-4D and FIG. 7).

The fluid collection region 124 can include an outer absorbent material configured to absorb a desired amount of fluid when placed in contact with the material. A variety of suitable absorbent materials can be selected so long as the material is biologically compatible, does not interact adversely with samples to be collected, absorbs and expels a desired amount of fluid, and/or is mechanically stable for the described uses. A suitable material can include an open cell polyurethane foam such as AQUAZONE® polyurethane or other similar relatively stable and inert foam material suitable for human application.

As shown, the supporting structure 122 is an elongated stick or rod. As shown, it can include a substantially flat structure that corresponds approximately to the gap shape of the holding and expelling component 130. This can help ensure that the collection device 120 moves smoothly through the gap 133 without rotation or movement when transported. Further, the structure 122 can include a ridge 28 extending along its length. The ridge 28 can correspond to an opening 131 on the collection device to further ensure smooth movement.

More proximate the collection region 124, the supporting structure can include an angled section 26. The angled section can include sloped edges being sloped downward towards a proximal end 29 of the collecting device 120. The sloped edges can assist in pushing the tabs 132, 132′ outward to prevent damage to the absorbent material at the colleting region 124. FIG. 7 shows an alternative embodiment of the collection device 120, which includes an angled section 26′ having a larger thickness but a longer length to taper more gradually.

FIG. 4A is a side view of a device for collecting fluid in a fully assembled state, according to certain embodiments. FIG. 4B is a side view of the device of FIG. 4A with certain components separated to illustrate use of the device. FIG. 4C is another side view of the device of FIGS. 4A and 4B with certain components separated to further explain use of the device. FIG. 4D is another side view of the device of FIGS. 4A-4C illustrating removal of the fluid collection device to illustrate expulsion of collected fluid into a test tube.

The present disclosure further includes methods of using the disclosed devices. As shown in FIG. 4A, the device 100 is fully assembled, as it may be configured when provided to a user or after collection of a sample. The components may be separated as shown in FIG. 4B, and the collection device 120 can be used to collect fluids by contacting the fluid collection region 124 with fluids, e.g., oral fluids. After collection of fluids, the collection device can be attached to the cap 140 (FIG. 4C), or alternatively the cap can be left attached to the collection device during collection. The collection device 120 with cap 140 can be returned to the sample collection container 110 for storage or transport.

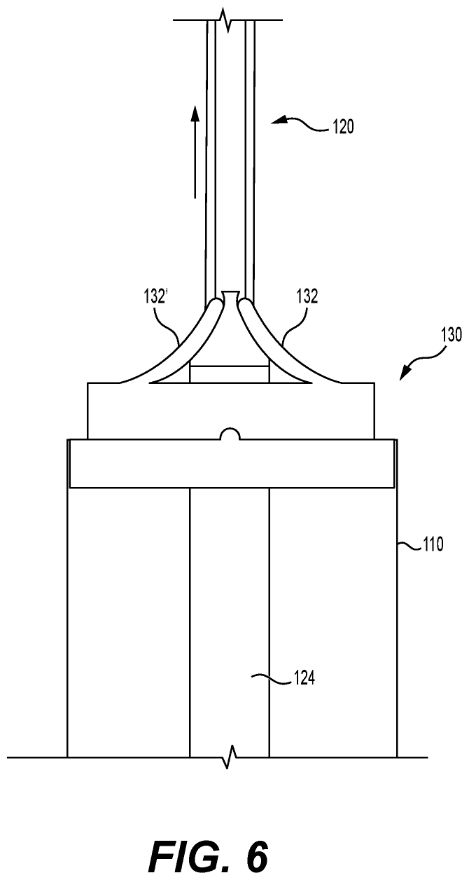

When it is time to use the sample, the collection device 120 can be removed, as shown in FIG. 4D. The collection device may be pulled upward such that the extension tabs 132, 132′ compress the absorbent material, as shown in FIG. 6.

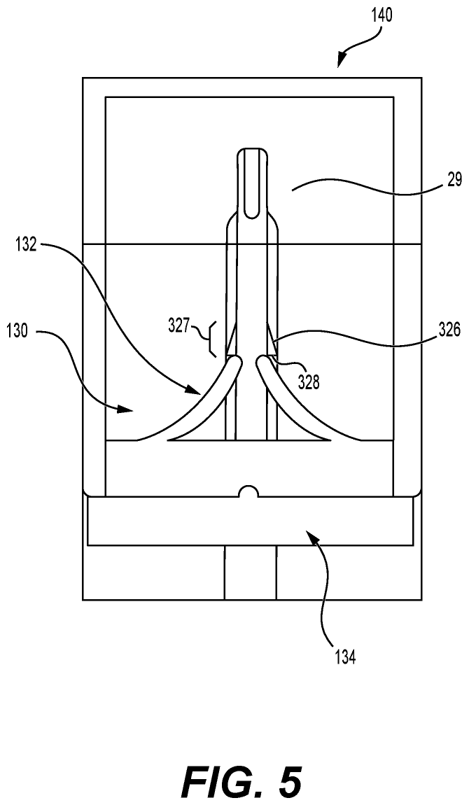

FIG. 5 is a side view of a portion of the fluid collection device of FIG. 1 illustrating a proximal portion of the fluid collection device while being held by the holding and expelling component. In some embodiments, the collection device 120 can includes a protrusion 326 near a proximal end 29. The protrusion can assist in securing the device 120 in the holding and expelling component 130. For example, as shown the protrusion 326 has a sloped or angled top portion 327 and flat bottom 328. The sloped portion 327 assists in passing the end 29 into the holding and expelling component 130 and the bottom portion 328 prevents the collection device from being easily removed by downward motion.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

1. A device for collection of fluid samples, comprising:
a fluid collection device, including:
an elongated supporting structure, and
a fluid collection region disposed at or near a distal end of the elongated supporting structure and including an absorbent material configured to collect fluids that are placed in contact with the absorbent material;
a holding and expelling component, including:
a base structure having an outer surface configured to contact a sidewall of a sample collection container, thereby securing the holding and expelling component in the sample collection container;
a first extension tab extending from a first side of a top portion of the base structure to a first end portion of the first extension tab; and
a second extension tab extending from a second side of a top portion of the base structure to a second end portion of the second extension tab;
wherein the first end portion and second end portion are arranged to form a gap therebetween, the gap sized to receive a proximal end of the elongated supporting structure to hold the elongated supporting structure; and
a sample collection container.

2. The device of claim 1, wherein the elongated supporting structure includes a substantially flat rod member.

3. The device of 1, wherein the first extension tab and the second extension tab are formed of a semi rigid material.

4. The device of claim 3, wherein the semi rigid material allows flexion of the first extension tab and second extension tab to apply force to a portion of the elongated supporting structure when the elongated supporting structure is placed in the gap.

5. The device of claim 1, wherein the gap is sized to allow the elongated supporting structure to move upward through the holding and expelling member, thereby applying compressive forces to sides of the fluid collection region as the fluid collection region is pulled through the gap.

6. The device of claim 1, wherein the fluid collection device includes a protrusion near a proximal end of the elongated supporting structure.

7. The device of claim 6, wherein the protrusion includes a sloped top edge and a substantially flat bottom portion, the sloped top edge configured to facilitate passage of the elongated supporting member structure through the gap in the holding and expelling component, and the substantially flat bottom portion configured to facilitate securing the fluid collection device in the holding and expelling component.

8. The device of claim 1, wherein the fluid collection device includes a protrusion forming an angled section above the absorbent material.

9. The device of claim 8, wherein the angled section is configured to facilitate passage of the elongated supporting member structure through the gap in the holding and expelling component.

10. The device of claim 1, wherein the first end portion of the first extension tab and the second end portion of the second extension tab are substantially parallel such that the gap is substantially linear.

11. The device of claim 10, wherein the gap is between about 0.25 mm and 3 mm in width.

12. The device of claim 11, wherein the gap is between about 0.0.5 mm and 2 mm in width.

13. The device of claim 1, wherein the elongated supporting structure fluid collection region has a thickness along its main body between about 7 and 12 mm.

14. A device for collection of fluid samples, comprising:
a fluid collection device, including:
an elongated supporting structure, and
a fluid collection region disposed at or near a distal end of the elongated supporting structure and including an absorbent material configured to collect fluids that are placed in contact with the absorbent material;
a holding and expelling component, including:
a base structure having an outer surface configured to contact a sidewall of a sample collection container, thereby securing the holding and expelling component in the sample collection container;
extension regions extending from the base structure and forming a holding and expelling portion, the holding and expelling portion configured to support a distal end of the elongated supporting structure, and upon pulling the fluid collection device through the holding and expelling portion, applying compressive force to the fluid collection region to cause expulsion of a desired amount of fluid from the absorbent material; and
a sample collection container.

15. The device of claim 14, wherein the desired amount of fluid comprises at least 30% of the fluid capable of being absorbed by the absorbent material.

16. The device of claim 14, wherein the desired amount of fluid comprises at least 40% of the fluid capable of being absorbed by the absorbent material.

17. The device of claim 14, wherein the desired amount of fluid comprises at least 50% of the fluid capable of being absorbed by the absorbent material.

18. The device of claim 14, wherein the elongated supporting structure includes a substantially flat rod member.

19. The device of claim 14, wherein extension regions are formed of a semi rigid material.

20. The device of claim 19, wherein the semi rigid material allows flexion of the extension regions to apply force to a portion of the elongated supporting structure when the elongated supporting structure is placed in the gap.

21. The device of claim 14, wherein the fluid collection device includes a protrusion near a proximal end of the elongated supporting structure.

22. The device of claim 21, wherein the protrusion includes a sloped top edge and a substantially flat bottom portion, the sloped top edge configured to facilitate passage of the elongated supporting member structure through the gap in the holding and expelling component, and the substantially flat bottom portion configured to facilitate securing the fluid collection device in the holding and expelling component.

23. The device of claim 14, wherein the fluid collection device includes a protrusion forming an angled section above the absorbent material.

24. The device of claim 23, wherein the angled section is configured to facilitate passage of the elongated supporting member structure through the gap in the holding and expelling component.

25. The device of claim 14, wherein the gap is substantially linear.

26. The device of claim 25, wherein the gap is between about 0.25 mm and 3 mm in width.

27. The device of claim 25, wherein the gap is between about 0.0.5 mm and 2 mm in width.

28. The device of claim 14, wherein the elongated supporting structure fluid collection region has a thickness along its main body between about 7 and 12 mm.

29. A device for collection of fluid samples, comprising:
an elongated supporting structure, and
a fluid collection region disposed at or near a distal end of the supporting structure and including an absorbent material configured to collect fluids that are placed in contact with the absorbent material;
wherein the fluid collection device includes a protrusion forming an angled section above the absorbent material.

30. A device for holding and expelling of fluid samples from a collection device, comprising:
a holding and expelling component, including:
a base structure having an outer surface configured to contact a sidewall of a sample collection container, thereby securing the holding and expelling component in the sample collection container;
a first extension tab extending from a first side of a top portion of the base structure to a first end portion of the first extension tab; and
a second extension tab extending from a second side of a top portion of the base structure to a second end portion of the second extension tab;
wherein the first end portion and second end portion are arranged to form a gap there between, the gap sized to receive a proximal end of the elongated supporting structure to hold the elongated supporting structure.
